(19) United States
(12) Patent Application Publication
(10) Pub. No.: US 2015/0075371 A1
(43) Pub. Date: Mar. 19, 2015

(54) IONIZATION AIR PURIFICATION SYSTEM FOR THE PASSENGER CABIN OF A VEHICLE

(71) Applicant: Clean Air Group, Inc., Fairfield, CT (US)

(72) Inventor: Anthony M. Abate, Seymour, CT (US)

(21) Appl. No.: 14/385,346

(22) PCT Filed: Mar. 15, 2013
(86) PCT No.: PCT/US13/32163

§ 371 Date: Sep. 15, 2014

Related U.S. Application Data
(60) Provisional application No. 61/611,676, filed on Mar. 16, 2012.

(51) Int. Cl. A61L 9/22; B03C 3/68

(52) U.S. Cl. CPC A61L 9/22; B03C 3/68; B03C 2201/30
USPC 95/2; 96/18; 95/26

(57) ABSTRACT

An ionization air purification system for the passenger cabin of vehicles, which modifies the degree of ionization by modifying the energy levels applied to the ionization tube or ionization source proportional to the change in air flow dynamics and air quality. In one embodiment, an ionization air purification system for the passenger cabin of a vehicle is disclosed. The system includes an ionization device for purifying the air prior to entering into the passenger cabin of the vehicle; and means for modifying the degree of ionization by modifying the energy levels applied to the ionization tube or ionization source proportional to the change in air flow dynamics and/or air quality.

### CROSS REFERENCE TO RELATED APPLICATION

This application claims the benefit of U.S. Provisional Application No. 61/611,676, filed Mar. 16, 2012, which is hereby incorporated by reference.

### FIELD OF THE INVENTION

The present invention relates to the use of air purification ionization systems in the field of transportation including, without limitation, cars, buses, trains, airplanes, trucks and other vehicles by which people and/or animals travel.

### BACKGROUND OF THE INVENTION

Systems that employ ionization as a means for removing contaminants, both solid and gaseous from an air stream are known. One of the most popular uses of the technology is in products offered by ATMOSAIR™, both stand alone systems such as towers as well as systems that are installed within HVAC systems in homes and commercial buildings. As well, technology particularly suitable for the ionization process is more fully described in U.S. Patent Application Publication No. US 2010-0247389 A1 entitled Bipoloar Ionization Tube owned by the assignee of this invention and whose disclosure is also incorporated by reference herein.

It is believed that there are no commercially available systems that address problems unique to passenger vehicles which include variable air flow parameters which are subject to significant variation in air flow and the significant, but often varying changes in air quality, particularly with regard to gaseous pollutants which are often encountered, all of which can have significant impact on the efficiency and efficacy of the ionization system.

FIG. 1 depicts in general schematic form the air flow system within a typical passenger automobile, wherein the air flow passes through a cabin air filter 1 prior to entering the passenger cabin.

Referring to FIG. 2 in which a typical passenger bus air circulation system 10 is schematically depicted, as is often the case with passenger buses or larger land vehicles, the air handler is larger and is often mounted in the rear of or on the roof 11 of the bus as shown.

As illustrated in FIG. 3, a typical passenger train air conditioning system 30 is illustrated, which is not dissimilar from that described with regard to a passenger bus. Various sensors determine air flow and air quality and whether the system is in a fresh air or air circulation mode.

Referring next to FIG. 4, the air circulation system in most passenger planes generally has air flow controls which can be turned on or off—or modified as to the degree of flow—by individual passengers at their seats. Once the plane is airborne the system is generally a recirculation system. Indeed there is much discussion in literature that the constant recirculation of unpurified air within air craft has had adverse affects upon the health of passengers as they are constantly exposed to the same recirculated air.

Although the potential benefits of an ionization system in the field of transportation would have many benefits, to date no one has created a system particularly suitable for such use.

### SUMMARY OF THE INVENTION

According to the present invention the unfulfilled need and the shortcomings in existing devices have been solved through the use of a system, method and apparatus, which modifies the degree of ionization by modifying the energy levels applied to the ionization tube or ionization source proportional to the change in air flow dynamics and/or air quality.

In one embodiment, an ionization air purification system for the passenger cabin of a vehicle is disclosed. The system includes an ionization device for purifying the air prior to entering into the passenger cabin of the vehicle; and means for modifying the degree of ionization by modifying the energy levels applied to the ionization tube or ionization source proportional to the change in air flow dynamics and air quality.

In another embodiment, a method for purifying air circulating within the passenger cabin of a vehicle is disclosed. The method includes the following steps: (i) passing the air stream through an ionization device for removing contaminants prior to entering into the passenger cabin of the vehicle; and (ii) modifying the degree of ionization by modifying the energy levels applied to the ionization tube or ionization source proportional to the change in air flow dynamics and air quality.

In another embodiment, a method of removing microbial contaminants from a cabin air filter without removing the filter from the vehicle is disclosed. The method includes the following steps: (i) initiating a program that allows an ionization device to operate for a predetermined time period with the car and blower off to allow a concentration of ions to interact with the microbial contaminants in the cabin air filter; and (ii) terminating the program after the predetermined time period has ended.

Although the invention is described specifically with regard to transportation such as cars, trucks, buses, trains and planes, it would as well have applicability to other environments having to address similar variations in air flow or air quality.

The systems described can either be self contained or incorporated within existing systems.

### BRIEF DESCRIPTION OF THE DRAWINGS

FIG. 1 depicts in general schematic form the air flow system within a typical passenger automobile.

FIG. 2 depicts an air flow system as might be found typically in a passenger bus.

FIG. 3 depicts schematically an air flow system as may be encountered typically in a passenger train.

FIG. 4 depicts an air flow system as typically found in a passenger aircraft.

FIG. 5 is a depiction of a plenum chamber such as might be found in a passenger vehicle, such as a car or truck, in which an ionization tube or like device is mounted, in accordance with at least one embodiment of the invention.

FIG. 6 is a schematic showing the electronic arrangement of FIG. 5.

FIGS. 7A-7C show alternate embodiments of that illustrated in FIG. 5.

FIG. 8 is a depiction of a typical passenger bus ventilation system, in which an ionization tube or like device is mounted, in accordance with the present invention.

FIG. 9 is a depiction of a typical passenger train ventilation system, in which an ionization tube or like device is mounted, in accordance with the present invention.

FIG. 10 is a depiction of a typical passenger plane ventilation system, in which an ionization tube or like device is mounted, in accordance with the present invention.

FIG. 11 illustrates an example of a suitable ionization self contained unit that can be used in certain embodiments of the invention.

### DETAILED DESCRIPTION OF PREFERRED EMBODIMENTS OF THE INVENTION

Without regard to the type of transportation under consideration each type typically includes an air intake system with or without a physical filter; a plenum chamber for the receipt of the air being taken in; a damper or other closure which either seals off the intake of outside air or permits the use of recirculated air; and air control fans or other devices which circulate the air through a series of ducts which eventually have outlets so as to provide the air to enter into the passenger cabin of the vehicle.

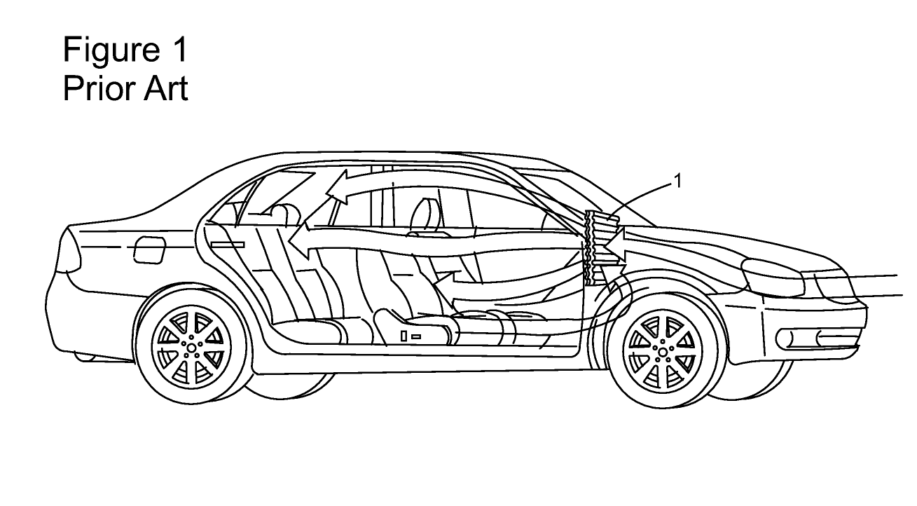

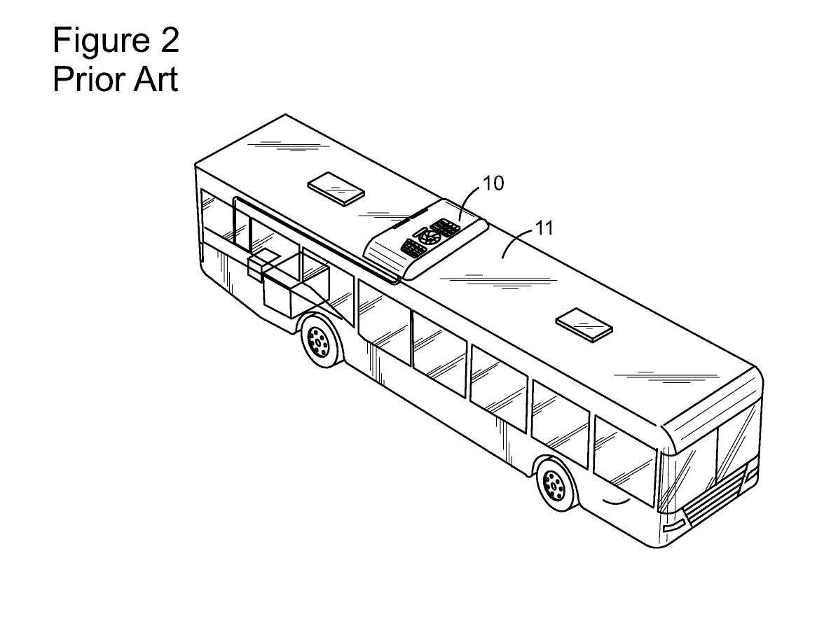

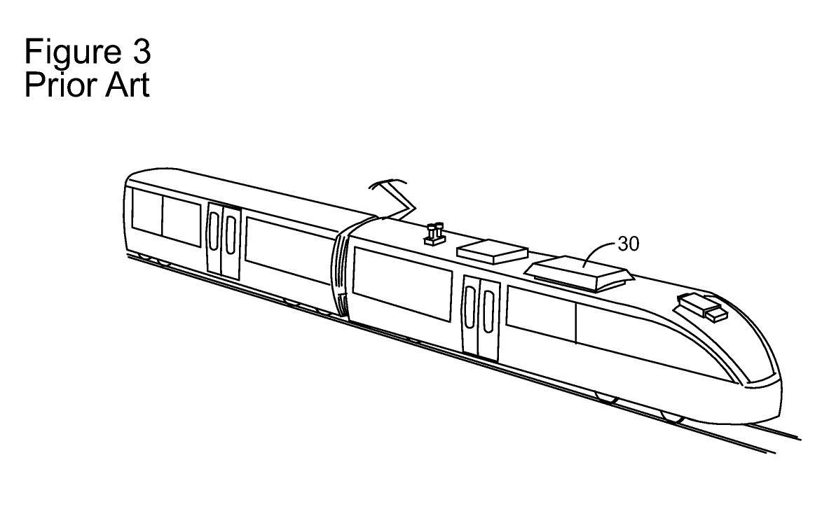

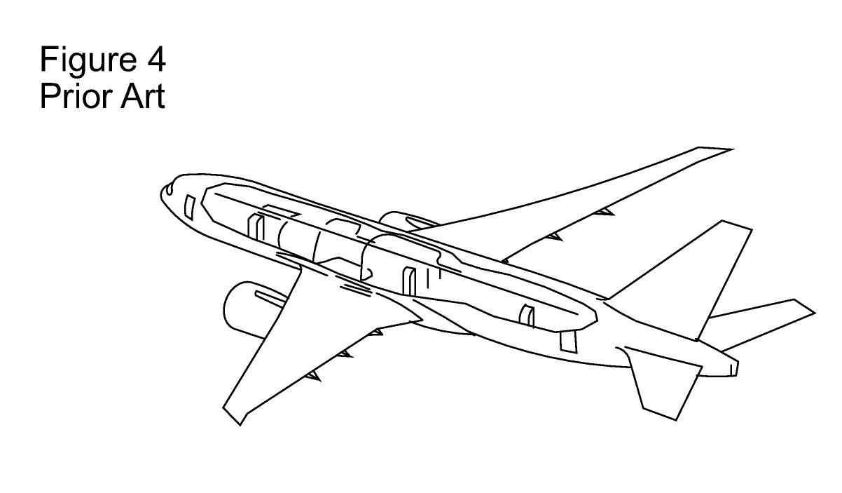

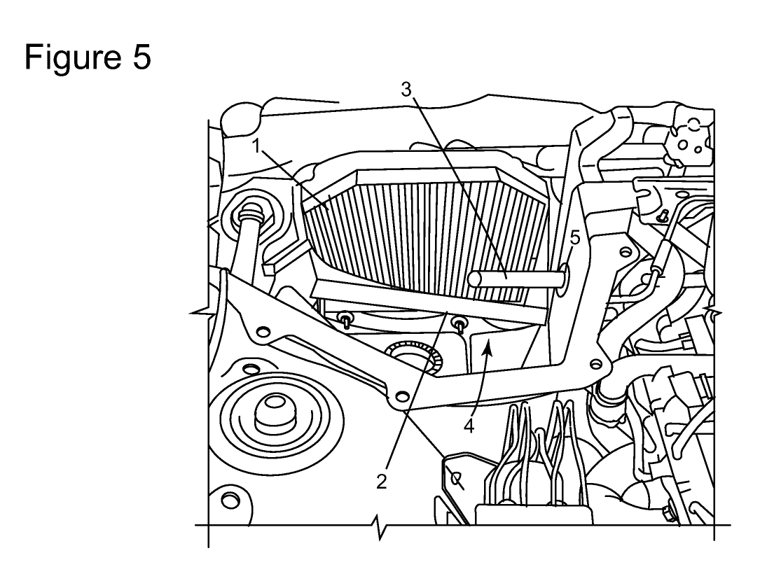

FIG. 5 illustrates the present invention, as implemented into the engine compartment of an automobile. As illustrated, a plenum chamber 4 is positioned within the engine compartment of the automobile, and is shown with the cover panel (not shown) removed. An air ionization tube 3 is fixedly mounted to a sidewall of the plenum chamber 4 so as to extend into the air flow from the intake, which in the case of an automobile (as shown in FIG. 1) generally is located along the base of the windshield as that location tends to reduce the extent of the intake of pollution in the outside air. The cabin air filter 1 is shown positioned against a sidewall of the plenum chamber 4 towards the passenger cabin.

As further illustrated in FIG. 5, the air ionization is performed using an ionization tube 3, but it is also contemplated that the ionization may be accomplished through other known forms of ionization generation. As illustrated, the ionization tube 3 is in turn secured via a socket assembly 5 that is mounted to a sidewall of the plenum chamber 4 and is electrically coupled generally via hard wiring to the 12 volt system that operates in the vehicle. A control device modifies the degree of ionization by modifying the energy levels applied to the ionization tube or ionization source proportional to the change in air flow dynamics and air quality.

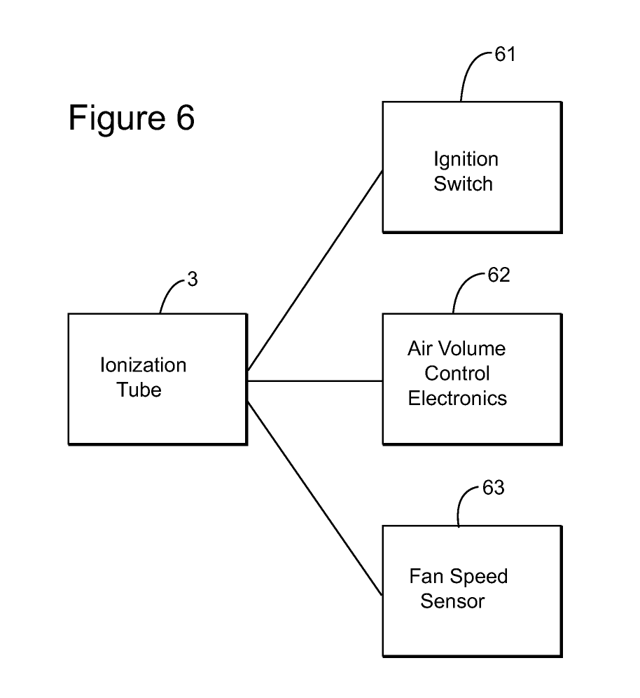

Referring to FIG. 6, the electronics are so configured that the ionization tube 3 is controlled by the ignition switch 61, such that it is only activated upon the ignition of the vehicle and deactivated when the vehicle is turned off. As well, the ionization system is connected to the manual or automatic electronics 62 that control the volumes of air that are emitted into the vehicle—generally ranging from a low to a high speed. In addition, there is a sensor 63 which acts in the nature of a rheostat (or like device) which senses the fan speed being commanded and changes the amount of power being delivered to the tube so as to adjust the level of ionization to be commensurate with the air flow.

Referring again to FIG. 5, as is conventional, there is usually a damper (not shown) that closes off the air flow from the plenum chamber 4 from the interior of the vehicle. As a result, outside air is not supplied to the passenger cabin. Instead, the air within the vehicle is being recirculated. Although the recirculated air has been treated, the closing of the damper will as well seal off the ionization tube from the air flow. Preferably the closure of the damper will as well cause a switch (not shown) to shut down the power to the ionization tube.

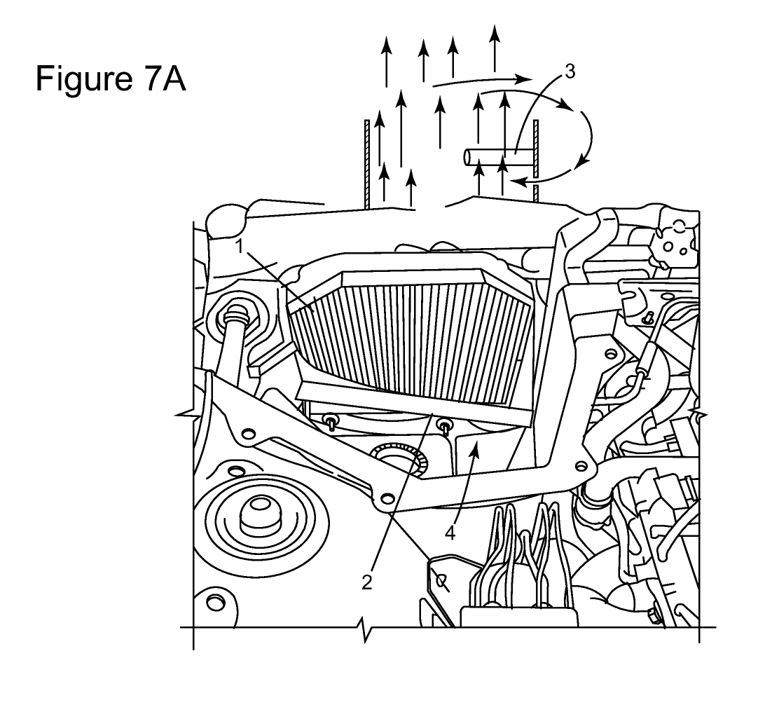

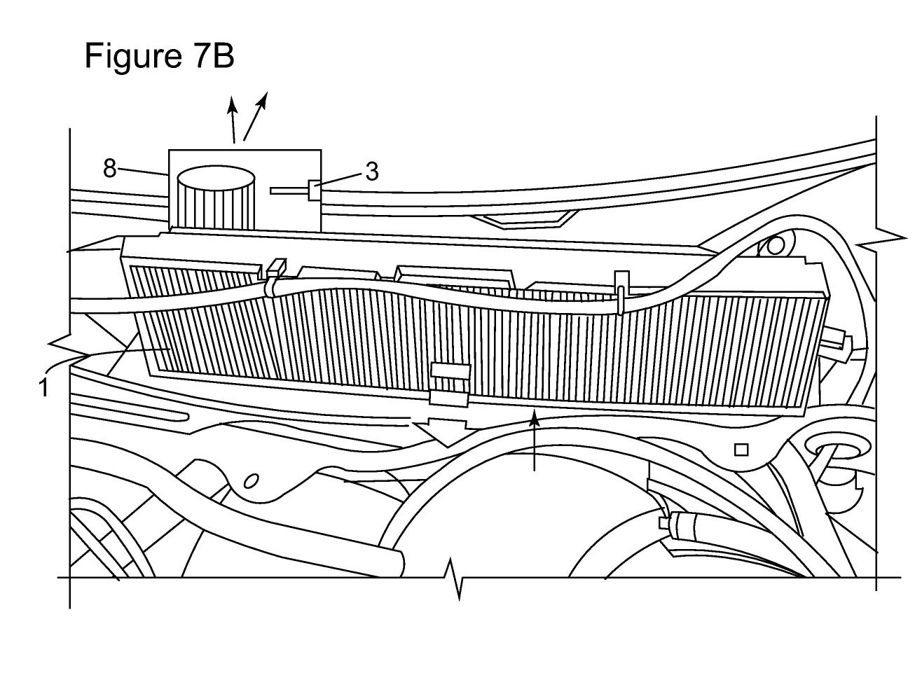

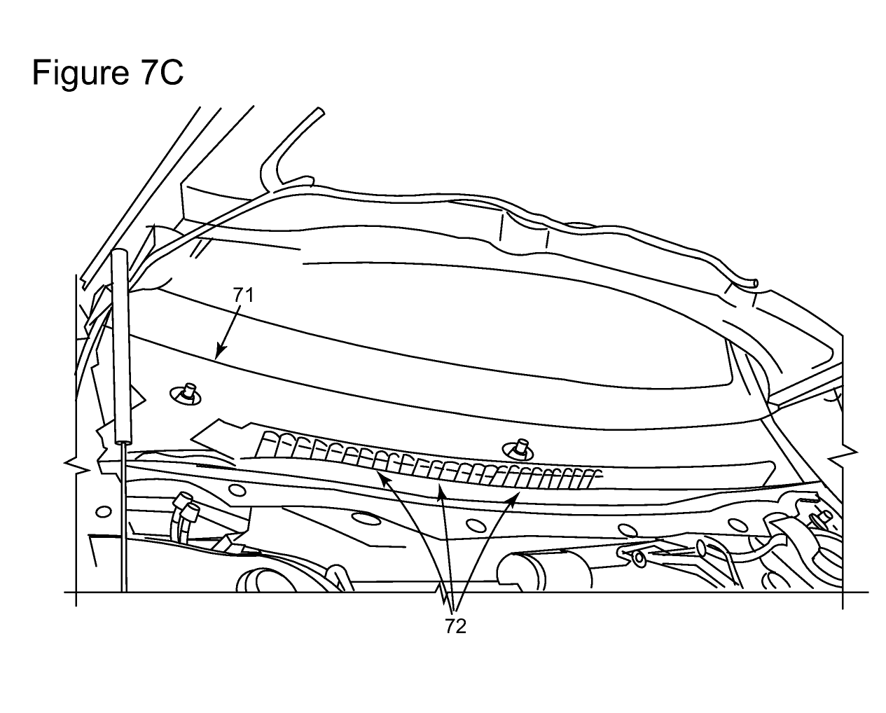

Referring to FIGS. 7A-7C, alternative embodiments are shown. Where it is desired that the ionization continue to operate for the recirculated air, the ionization tube 3, rather than be mounted in the plenum chamber 4, is mounted in an auxiliary mounting chamber 8 (see FIG. 7B) or within a main air circulation duct 6 (see FIG. 7A) which is downstream from the damper (not shown) and within the recirculating air flow stream. As shown in FIG. 7C, a cowl grill 71 is positioned near the passenger cabin of the vehicle and includes an outside air intake 72 that acts as an inlet for the air conditioning system of the vehicle.

Preferably the control system for the power to the ionization tube in the recirculation mode is reduced so as to lessen the degree of ionization to be proportional to the desired degree of ionization, given that the air being circulated has been preconditioned but nonetheless may be subject to gaseous contaminants present in the interior of the vehicle such as, for example, as might be encountered by smoking.

In newer passenger cars, cabin air filters have become increasingly standard. These are typically pleated media filters 1 as shown in FIG. 5. These filters are intended to trap airborne contaminants from the air entering the car. Mold and bacteria grow on these over time especially since outside air can be laden with moisture which can foster microbial growth. In another embodiment, the present invention can offer a “clean mode” feature that would allow the unit to operate for a set period of time with the car and blower off to allow a concentration of ions to interact with the microbial contaminants in the cabin air filter. This can be a user enabled function. An automated message on the car information screen can remind the car owner to run this “clean mode” feature.

As discussed below, the present invention can be implemented into other types of passenger vehicles including, without limitation, buses, trains, and airplanes.

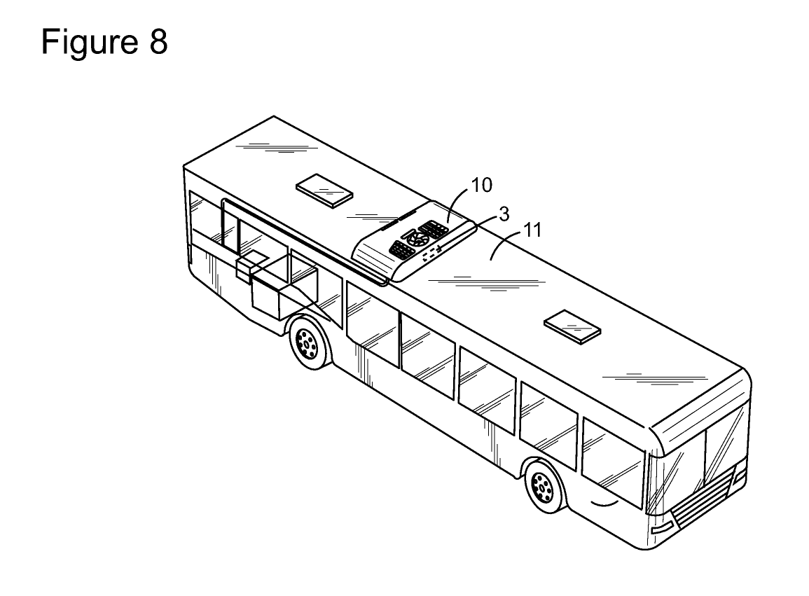

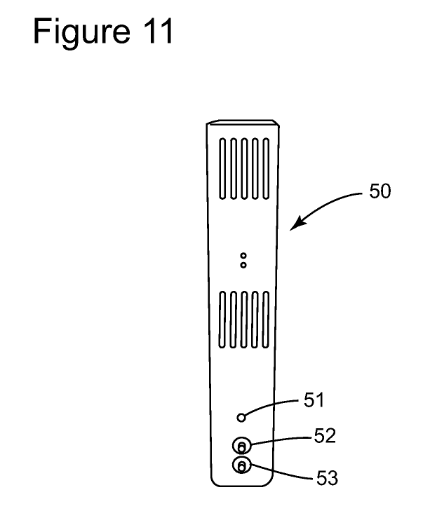

Referring to FIG. 8, the ionization tube 3 of the present invention can be positioned within the air circulation system 10, located on the roof 11 of a passenger bus. Although the air flow and desired ionization environment employed is substantially the same as that described with regard to passenger cars, given the size of the interior of a passenger bus, train, or airplane and the likelihood that it would be in operation for a continuous extended period of time with a greater likelihood of encountering physical and gaseous contaminants, a preferable system for buses, trains, or airplanes includes the mounting of a series of ionization self contained units along, for example, the upper side walls above the windows or along the interior ceiling of the bus, train or airplane. One such example of a suitable ionization self contained unit 50 is illustrated in FIG. 11, which includes a power indicator 51, an ion level control 52, and a fan speed control 53. The controls can be automated into the system in one embodiment. In another embodiment, the controls can be operated by the driver through a centralized control unit. An exemplary commercially available ionization unit is sold by the assignee of the present invention as model number ATMOSAIR T400WM, currently available at www.atmosair.com.

Although the individual units are self contained, they are coupled with the 12 volt system of the bus (although an auxiliary power supply could also be used). In the preferred embodiment, the same general type of sensing devices in terms of air flow and air quality described with respect to cars is employed to modify the amount of ionization for each of the units, either individually or collectively.

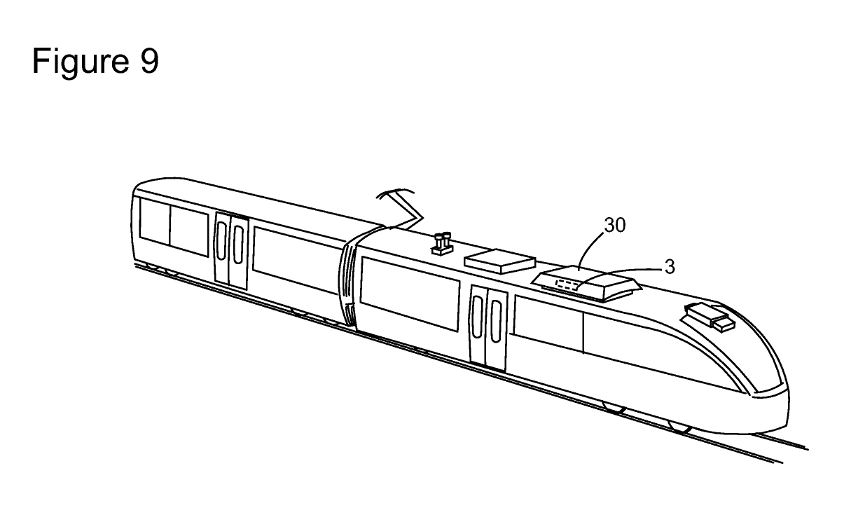

As illustrated in FIG. 9, a system, not dissimilar from that described with regard to the buses is illustrated for passenger trains. Various sensors determine air flow and air quality and whether the system is in a fresh air or air circulation mode. The ionization tube 3 of the present invention can be positioned within the air circulation systems 30.

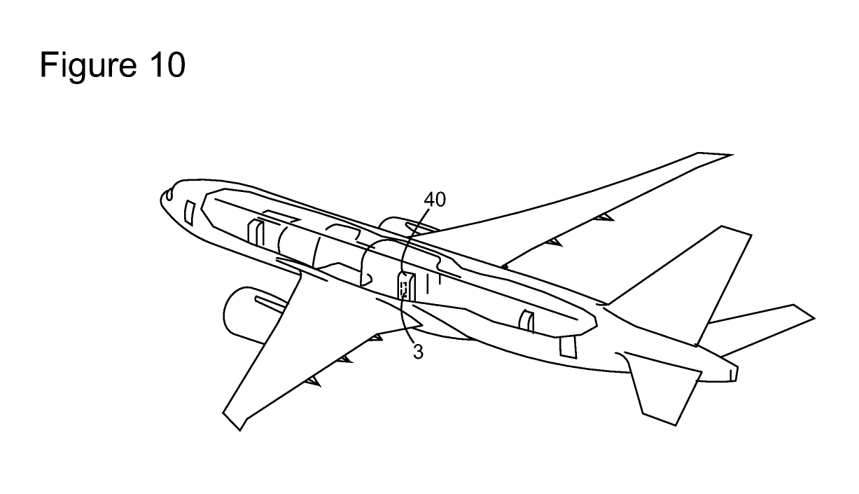

Referring next to FIG. 10, the air circulation system in most passenger planes generally has air flow controls which can be turned on or off—or modified as to the degree of flow—by individual passengers at their seats. Once the plane is airborne the system is generally a recirculation system. Indeed there is much discussion in literature that the constant recirculation of unpurified air within air craft has had adverse affects upon the health of passengers as they are constantly exposed to the same recirculated air. The ionization tube 3 of the present invention can be positioned within one or more bulkhead ventilation units 40 for purifying the air.

Thus, the systems previously described can be used within the plane, but the preferable system has the ionization tube or multiple tubes or other ionization source mounted within the ducts that feed the exit orifices, are downstream from the initial air intake and downstream from the damper or cutoff valving that closes off the air circulation from the outside sourcing of air, but upstream from the air shut off valves at the passengers' seats.

It should be understood that the present invention can be used in a passenger vehicle both while it is stationary and while it is moving.

The above disclosure is intended to be illustrative and not exhaustive. This description will suggest many modifications, variations, and alternatives may be made by ordinary skill in this art without departing from the scope of the invention. Those familiar with the art may recognize other equivalents to the specific embodiments described herein. Accordingly, the scope of the invention is not limited to the foregoing specification.

What is claimed is:

1. An ionization air purification system for the passenger cabin of a vehicle comprising:
an ionization device for purifying the air prior to entering into the passenger cabin of the vehicle; and
a control device for modifying the degree of ionization by modifying the energy levels applied to the ionization device proportional to a change in air flow dynamics and/or air quality.

2. The ionization air purification system of claim 1, wherein the vehicle is a passenger automobile.

3. The ionization air purification system of claim 2, wherein the ionization device is mounted within a plenum chamber located within the engine compartment of the passenger automobile.

4. The ionization air purification system of claim 2, wherein the ionization device is mounted within an auxiliary mounting chamber that is adapted for use with a recirculating air flow stream within the passenger cabin.

5. The ionization air purification system of claim 1, wherein the vehicle is a passenger bus.

6. The ionization air purification system of claim 1, wherein the vehicle is a passenger train.

7. The ionization air purification system of claim 1, wherein the vehicle is a passenger plane.

8. The ionization air purification system of claim 1, wherein the ionization device is an ionization tube.

9. The ionization air purification system of claim 8, wherein the ionization tube is electrically coupled to an on-board electrical system that operates in the vehicle.

10. The ionization air purification system of claim 9, further comprising a switch for selectively activating the ionization device.

11. The ionization air purification system of claim 1, wherein the control device includes a sensor which senses the air flow dynamics and modifies the amount of power being delivered to the ionization device according to a predetermined ratio.

12. The ionization air purification system of claim 1, wherein the control device includes a sensor which senses the air quality and modifies the amount of power being delivered to the ionization device according to a predetermined ratio.

13. A method for purifying air circulating within the passenger cabin of a vehicle comprising:
passing the air stream through an ionization device for removing contaminants prior to entering into the passenger cabin of the vehicle; and
modifying the degree of ionization by modifying the energy levels applied to the ionization device proportional to a change in air flow dynamics and air quality.

14. A method of removing microbial contaminants from a cabin air filter without removing the filter from the vehicle comprising:
initiating a program that allows an ionization device to operate for a predetermined time period with the car and blower off to allow a concentration of ions to interact with the microbial contaminants in the cabin air filter; and
terminating the program after the predetermined time period has ended.
